(19) United States
(12) Patent Application Publication
(10) Pub. No.: US 2004/0052736 A1
(43) Pub. Date: Mar. 18, 2004

(54) Dentifrice compositions comprising talc

(75) Inventors: Lijiang Yang, Beijing (CN); Jiang Yue, Beijing (CN); Yunbin Fei, Beijing (CN); Yun Wang, Beijing (CN); Xiujun Xu, Beijing (CN)

Correspondence Address: THE PROCTER & GAMBLE COMPANY, INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company, Cincinnati, OH

(21) Appl. No.: 10/618,472

(22) Filed: Jul. 11, 2003

Related U.S. Application Data
(60) Provisional application No. 60/410,094, filed on Sep. 12, 2002.

(51) Int. Cl. A61K 7/18

(52) U.S. Cl. 424/52

(57) ABSTRACT

The present invention is directed to a dentifrice composition comprising from about 1% to about 50% by weight of talc; a fluoride salt that provides from about 1 ppm to about 3000 ppm by weight of a fluoride ion; from about 5% to about 50% by weight of an abrasive polishing material; and from about 30% to about 90% of one or more aqueous carriers. The dentifrice composition has a pH of about 8 or above at 25° C.

### CROSS REFERENCE TO RELATED APPLICATION

This application claims the benefit of U.S. Provisional application No. 60/410094, filed on Sep. 12, 2002.

### FIELD OF THE INVENTION

The present invention is directed to dentifrice compositions. More particularly, the present invention is directed to dentifrice compositions comprising talc.

### BACKGROUND OF THE INVENTION

It is commonly recognized that the presence of small amounts of fluoride occurring naturally in drinking water has a pronounced effect in reducing the incidence of dental caries in permanent teeth of children consuming such water from birth through eight years of age. Fluoride salts have been introduced into public water supplies in many communities with similar results. This method of dental caries prophylaxis is not available, however, to large numbers of people whose drinking water is obtained from small, private, fluoride-deficient sources such as individual wells and the like. Further, the addition of fluoride to common public water sources is not always accepted or permitted.

Topical application of aqueous fluoride solutions by dentists or dental hygienists likewise provide an excellent measure of protection against dental caries. Various fluoride compounds have been employed in this manner, including stannous fluoride and sodium fluoride. Another method of employing the anticariogenic properties of fluoride salts comprises incorporating such materials with a compatible abrasive to form a prophylactic paste composition for use by dentists or dental hygienists on a professional basis.

Limitation on the availability of fluoride therapy by way of water supply or professional treatment has led to extensive efforts to incorporate fluoride salts in oral compositions for use in the home in the form of fluoride-containing dentifrices. Although effective dental caries protection has been obtained through the use of the aforementioned fluoride-containing compounds, such compounds are not always stable when they are formulated with some other ingredients that are readily available for use in dentifrice compositions, such as talc.

Talc has been used in dentifrices as an abrasive and formulation rheology modifier in combination with other abrasives such as calcined kaolin, see U.S. Pat. No. 4,428,928. However, when formulating talc into many types of fluoride-containing dentifrices, the talc reacts with the fluoride, causing a decrease in the efficacy of the soluble fluoride ion. Thus, there remains a need to provide fluoride-containing dentifrice compositions comprising talc in which there is sufficient compatibility between the talc and the fluoride such that there is no marked decrease in fluoride (anti-caries) efficacy.

### SUMMARY OF THE INVENTION

The present invention is directed to dentifrice compositions comprising from about 1% to about 50% by weight of a talc; a fluoride salt that provides from about 1 ppm to about 3000 ppm by weight of a fluoride ion; from about 5% to about 50% by weight of an abrasive polishing material; and from about 30% to about 90% of one or more aqueous carriers. The dentifrice composition has a pH of about 8 or above at 25° C.

### DETAILED DESCRIPTION OF THE INVENTION

As used herein, “comprising” means that other steps and other ingredients which do not affect the end result can be added. This term encompasses the terms “consisting of” and “consisting essentially of”.

(a) Talc

Talc, with chemical name of “Hydrous Magnesium Silicate”, is an essential component of the dentifrice composition of the present invention. Any type of talc (for example, a natural talc, a synthesized talc or a modified talc) can be used for the dentifrice composition of the present invention. A preferred talc is a natural talc, for example, natural talc of the USP grade, which tends to be low in cost and readily available.

Additional talcs that are suitable for use in the present invention include, but are not limited to, methicone treated talc which is provided by Miyoshi Kasei Inc., Saitama-city, Saitama, Japan. Other talcs useful herein are high purity hydrotalcites which are stratiform, anionic mixed metal hydroxides of the general formula

M₂ₓ²⁺M₂³⁺(OH)_{4x+4.}A_{2/n}ⁿ⁻.zH₂O

wherein M₂ₓ²⁺, M₂³⁺ are divalent and trivalent metal(s) respectively, x ranges from 0.5 to 10 in intervals of 0.5, A is an interstitial anion selected from the group consisting of a hydroxide ion and an organic ion, n is the charge of the interstitial anion, and z is an integer of 1 to 6. Such hydrotalcites are described in PCT patent publication No. WO 96/23727, which is assigned to LVEDEA AG, published on Aug. 8, 1996 (corresponding to U.S. Pat. Nos. 6,517,795; 6,514,473; 6,180,764; and US publication no. 20010001653A1).

The level of talc in the dentifrice composition of the present invention is from about 1% to about 50%, preferably, from about 5% to about 25% by weight, and more preferably from about 5% to about 15%.

(b) Fluoride Salt

Fluoride salt is also an essential component of the dentifrice of the present invention. Preferably the fluoride salt is selected from the group consisting of sodium fluoride, aluminum fluoride, stannous fluoride, potassium fluoride, zinc fluoride, sodium fluorophosphates and mixtures thereof. More preferably, the fluoride salt is a sodium fluoride. Other suitable fluoride salts are described in U.S. Pat. No. 4,108,979 to Muhler, et al., issued Aug. 22, 1978.

The method of measuring the fluoride ion present in the compositions herein is as follows (China National Standard: GB8372-2001): prepare a homogeneous toothpaste slurry of the dentifrice of the present invention and water with the weight ratio of the dentifrice to the water being about 1:4 and centrifuge the slurry to get clear supernatant. Mix the supernatant with citrate buffer thoroughly; apply the mixture to a calibrated fluoride ion-selective electrode (ISE) meter, e.g., Orion Fluoride-Specific Ion Combination Electrode (Orion Research, Inc., 500 Cummings Center, Beverly, Mass., USA) and obtain the fluoride concentration. In the present invention, the fluoride salt provides from about 1 ppm to about 3000 ppm, preferably, from about 10 ppm to about 1500 ppm, of fluoride ion. Also, the level of fluoride salt in the dentifrice composition of the present invention is selected according to its fluoride ion producing capability. The ion producing capability varies by the type of fluoride ion are selected. If using sodium fluoride, however, the preferred level of sodium fluoride salt in the dentifrice composition of the present invention is from about 0.001% to about 1%, more preferably, from about 0.005% to about 0.5% by weight.

(c) Abrasive Polishing Materials

The abrasive polishing materials useful herein may be selected from any material which does not excessively abrade dentine. The abrasive polishing material preferably has a calcium content of less than about 23%. Typical abrasive polishing materials include silicas including gels and precipitates; aluminas; phosphates including orthophosphates, polymetaphosphates, and pyrophosphates; and mixtures thereof. Specific preferred examples of the abrasive polishing material useful herein include silicas (further described below), dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, resinous abrasive materials such as particulate condensation products of urea and formaldehyde, dicalcium phosphate, alumina trihydrate, insoluble sodium metaphosphate, and others such as disclosed by Cooley, et al., in U.S. Pat. No. 3,070,510, issued Dec. 25, 1962. Mixtures of abrasives may also be used.

Silica dental abrasives are highly preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, “Zeodent”, particularly the silica carrying the designation “Zeodent 119.” The abrasive in the dentifrice compositions described herein is generally present at a level of from about 5% to about 50% by weight of the composition, preferably from about 7% to about 30% of abrasive, by weight of the oral composition.

(d) pH of the Compositions

The dentifrice compositions of the present invention have a pH of about 8 or above, preferably, from about 8 to about 10, more preferably, from about 8.5 to about 9.5, at 25° C. In the present invention, not wanting to be limited by theory, it is believed that because of the neutral or alkaline pH, compatibility of talc with fluoride salt in the present dentifrice compositions is provided.

The method of measuring the pH is as follows: prepare a homogeneous slurry of the dentifrice of the present invention and water with the weight ratio of the dentifrice to the water being about 1:3; apply the slurry to a calibrated potentiometric instrument glass electrode, e.g.,, Orion Ross Sure-Flow combination VWR #34104-830 (Orion Research Inc., 500 Cummings Center, Beverrly, Mass., USA); and obtain the pH value at 25° C.

(e) Chelating agent

It is believed that incorporation of a chelating agent may further improve the compatibility of talc with fluoride. Thus, it is desirable although not essential to add a chelating agent to the compositions herein. Preferred chelating agents are selected from the group consisting of phosphates, pyrophosphates, tripolyphosphates, citrates, carbonates, tartrates, acetates, diacetates, phytic acid, EDTA (disodium ethylenediamine tetraacetate), EHDP (disodium ethane-1-hydroxy-1,1-diphosphonate) and mixtures thereof. More preferably, phosphates, pyrophosphates, tripolyphosphates, and mixtures thereof are used. The level of the chelating agent in the dentifrice composition of the present invention is from about 0.1% to about 20%, preferably, from about 0.5% to about 10%. The chelating agents can be added into the dentifrice composition directly during the making process, or they can be premixed with talc initially to treat it before putting into the dentifrice composition.

(f) Aqueous Carriers

In preparing the present compositions, it is desirable to add one or more aqueous carriers to the compositions. Such materials are well known in the art and are readily chosen by one skilled in the art based on the physical and aesthetic properties desired for the compositions being prepared. Aqueous carriers typically comprise from about 30% to about 90% by weight of the oral composition, preferably from about 50% to about 85%.

The present compositions typically contain some thickening material or binders to provide a desirable consistency. Preferred thickening agents are carboxyvinyl polymers, carrageenan, hydroxyethyl cellulose, and water soluble salts of cellulose ethers such as sodium carboxymethylcellulose and sodium hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can be used in an amount of from about 0.1% to about 15%, by weight of the dentifrice composition.

Another optional component of the compositions desired herein is a humectant. The humectant serves to keep toothpaste compositions from hardening upon exposure to air and certain humectants can also impart desirable sweetness of flavor to toothpaste compositions. Suitable humectants for use in the invention include glycerin, sorbitol, polyethylene glycol, propylene glycol, and other edible polyhydric alcohols. The humectant generally comprises from about 0% to 70%, and preferably from about 5% to 50%, by weight of the composition.

Water employed in the preparation of commercially suitable oral compositions should preferably be of low ion content and free of organic impurities. The dentifrice composition will contain a water of from about 5% to about 70%, preferably from about 20% to about 65%, weight of the composition. The amounts of water include the free water which is added plus that which is introduced with other materials, such as with sorbitol, silica, surfactant solutions, and/or color solutions.

The present compositions may also comprise surfactants, also commonly referred to as sudsing agents. Suitable surfactants are those which are reasonably stable and foam throughout a wide pH range. The surfactant may be anionic, nonionic, amphoteric, zwitterionic, cationic, or mixtures thereof. Anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate and sodium coconut monoglyceride sulfonates are examples of anionic surfactants of this type. Other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. Nonionic surfactants which can be used in the compositions of the present invention can be broadly defined as compounds produced by the condensation of alkylene oxide groups (hydrophilic in nature) with an organic hydrophobic compound which may be aliphatic or alkyl-aromatic in nature. Examples of suitable nonionic surfactants include poloxamers (sold under trade name Pluronic), polyoxyethylene, polyoxyethylene sorbitan esters (sold under trade name Tweens), fatty alcohol ethoxylates, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides, and mixtures of such materials. The amphoteric surfactants useful in the present invention can be broadly described as derivatives of aliphatic secondary and tertiary amines in which the aliphatic radical can be a straight chain or branched and wherein one of the aliphatic substituents contains from about 8 to about 18 carbon atoms and one contains an anionic water-solubilizing group, e.g., carboxylate, sulfonate, sulfate, phosphate, or phosphonate. Other suitable amphoteric surfactants are betaines, specifically cocamidopropyl betaine. Mixtures of amphoteric surfactants can also be employed. Many of these suitable nonionic and amphoteric surfactants are disclosed by Gieske et al., U.S. Pat. No. 4,051,234, issued Sep. 27, 1977. The present composition typically comprises one or more surfactants each at a level of from about 0.25% to about 12%, preferably from about 0.5% to about 8%, by weight of the composition.

Titanium dioxide may also be added to the present composition. Titanium dioxide is a white powder which adds opacity to the compositions. Titanium dioxide generally comprises from about 0.25% to about 5%, by weight of the composition.

Coloring agents may also be added to the present composition. The coloring agent may be in the form of an aqueous solution, preferably 1% coloring agent in a solution of water. Color solutions generally comprise from about 0.01% to about 5%, by weight of the composition.

A flavor system can also be added to the compositions. Suitable flavoring components include, but are not limited to, oil of wintergreen, oil of peppermint, oil of spearmint, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, and mixtures thereof. A flavor system is generally used in the compositions at levels of from about 0.001% to about 5%, by weight of the composition.

Sweetening agents can be added to the compositions. These include saccharin, dextrose, sucrose, lactose, maltose, levulose, aspartame, sodium cyclamate, D-tryptophan, dihydrochalcones, acesulfame, and mixtures thereof. Various coloring agents may also be incorporated in the present invention. Sweetening agents and coloring agents are generally used in toothpastes at levels of from about 0.005% to about 5%, by weight of the composition.

The dentifrice compositions of the present invention have improved stability because of the better compatibility of the talc with fluoride and thus, providing anti-caries efficacy. Also, the dentifrice composition of the present invention has improved rheology properties and can be made to have a higher density than comparable formulations that do not comprise talc. Such higher density formulations can potentially be provided at overall lower cost to consumers, but without comprising the efficacy of the dentifrice, especially in terms of the fluoride salt and the abrasive polishing material.

### EXAMPLES 1-6

The following examples of dentifrice compositions further describe and demonstrate embodiments within the scope of the present invention. These examples are given solely for the purpose of illustration and are not to be construed as limitations of the present invention as many variations thereof are possible without departing from the spirit and scope. All levels of ingredients are by weight percentage of the dentifrice composition.

| Component | Ex. 1 | Ex. 2 | Ex. 3 | Ex. 4 | Ex. 5 | Ex. 6 |
|---|---|---|---|---|---|---|
| Sodium Fluoride | 0.20 | 0.31 | 0.31 | 0.31 | 0.31 | 0.31 |
| Talc | 10.0 | 10.0 | 10.0 | 10.0 | 10.0 | 10.0 |
| Sorbitol Solution | 45.0 | 30.0 | 30.0 | 30.0 | 10.0 | 10.0 |
| (70% solution) | | | | | | |
| Precipitated Silica | 15.0 | 20.0 | 15.0 | 20.0 | 10.0 | 10.0 |
| Sodium Alkyl Sulfate (27.9% | 7.5 | 7.5 | 7.5 | 7.5 | 7.5 | 5.0 |
| solution) | | | | | | |
| Trisodium Phosphate | 1.8 | 2.0 | 1.0 | 1.0 | 1.7 | 1.7 |
| Carboxymethycellulose, | 1.0 | 0.6 | 0.6 | 0.6 | 1.5 | 1.5 |
| Sodium | | | | | | |
| Carbomer 956 | 0.3 | 0.2 | 0.2 | 0.2 | 0.4 | 0.4 |
| Saccharin, Sodium | 0.25 | 0.25 | 0.25 | 0.25 | 0.25 | 0.25 |
| Titanium Dioxide, Rutile | 0.25 | 0.25 | 0.25 | 0.25 | 0.25 | 0.25 |
| Flavor | 0.9 | 0.9 | 0.9 | 0.9 | 0.9 | 0.9 |
| Trisodium Polyphosphate | — | — | — | 3.82 | — | — |
| Betaine 30% Solution | — | — | — | — | 2.0 | 2.0 |
| Treated Water | 17.8 | 27.99 | 33.99 | 25.17 | 55.19 | 57.69 |

The dentifrice compositions of the foregoing examples are prepared as follows.

Water and sorbitol are mixed into a making vessel and continuously stirred until full dispersion. Then, all salts and thickeners are added into vessel at the functioning of homogenizer. Next, talc and other abrasives (e.g., silica) are added into vessel and mixed at vacuum. Finally, sodium alkyl sulfate and flavor are added into vessel and mixed until full dispersion.

The fluoride ion concentration and pH of the dentifrice compositions may be measured as follows. 20.000 g of the dentifrice composition of Example 1 is weighed and then placed into a plastic flat bottomed container of approximately 50 mL capacity which has a tight fitting lid. The compositions is diluted with water to 100 mL and mixed thoroughly to obtain a homogeneous slurry. The mixture is centrifuged at 2000 rpm for 30 min to obtain a clear supernatant. 10 mL of the supernatant is mixed with 5 mL citrate buffer solution (containing 100 g sodium citrate, 60 mL acetic acid, 60 g sodium chloride, 30 g sodium hydrate and diluted by water to 1000 mL, adjust pH=5.0˜5.5) and the mixture is applied to Orion Fluoride-Specific Ion Combination Electrode (Cat. #9609N, Orion Research, Inc., 500 Cummings Center, Beverly, Mass., USA); then the fluoride concentration value is obtained. The fluoride ion concentration is about 30.5 ppm.

3.998˜4.002 g of the dentifrice composition of Example 1 is weighed and then placed into a glass beaker and add 11.998˜12.002 g deionized water is then added. Next, the mixture is stirred thoroughly with a stir bar to obtain a homogeneous slurry. The slurry is applied to a calibrated Orion Combination Electrode (VWR #34104-830, Orion Research Inc., 500 Cummings Center, Beverly, Mass., USA) at 25° C. to obtain the pH value. The obtained pH level is from about 7.66 to about 10.61.

What is claimed is:

1. A dentifrice composition comprising:
(1) from about 1% to about 50% by weight of a talc;
(2) a fluoride salt that provides from about 1 ppm to about 3000 ppm by weight of a fluoride ion;
(3) from about 5% to about 50% by weight of an abrasive polishing material; and
(4) from about 30% to about 90% of one or more aqueous carriers;
wherein the dentifrice composition has a pH of about 8 or above at 25° C.

2. The dentifrice composition according to claim 1 wherein the talc is selected from the group consisting of natural talcs, synthesized talcs, modified talcs, high purity hydrotalcites, and mixtures thereof.

3. The dentifrice composition according to claim 2 wherein the talc is natural talc.

4. The dentifrice composition according to claim 1 further comprising from about 0.1% to about 20% by weight of a chelating agent.

5. The dentifrice composition according to claim 4 wherein the chelating agent is selected from the group consisting of phosphates, pyrophosphates, tripolyphosphates, citrates, carbonates, tartrates, acetates, diacetates, phytic acid, EDTA, EHDP, and mixtures thereof.

6. The dentifrice composition according to claim 1, wherein the fluoride salt is selected from the group consisting of a sodium fluoride, aluminum fluoride, stannous fluoride, potassium fluoride, zinc fluoride, sodium fluorophosphates, and mixtures thereof.

7. The dentifrice composition according to claim 6, wherein the fluoride salt is a sodium fluoride.

8. The dentifrice composition according to claim 1 wherein the aqueous carriers are selected from the group consisting of thickeners, humectants, water, surfactants, titanium dioxide, coloring agents, flavor systems, sweeteners, and mixtures thereof.

9. The dentifrice composition according to claim 1 wherein the abrasive polishing material is a silica abrasive.
